(19) United States
(12) Patent Application Publication
(10) Pub. No.: US 2017/0360334 A1
(43) Pub. Date: Dec. 21, 2017

(54) Device and Method for Determining a State of Consciousness

(71) Applicant: Nokia Technologies Oy, Espoo (FI)

(72) Inventors: Niku OKSALA, Tampere (FI); Antti VEHKAOJA, Tampere (FI)

(21) Appl. No.: 15/528,178

(22) PCT Filed: Dec. 14, 2015
(86) PCT No.: PCT/FI2015/050879

§ 371 Date: May 19, 2017

(30) Foreign Application Priority Data
Dec. 12, 2014 (FI) 20146088

(51) Int. Cl. A61B 5/11; A61B 5/0205; A61B 5/0402; A61B 5/00; A61B 5/01; A61B 5/145; A61B 5/024; A61B 5/0245; A61B 5/021; A61B 5/08

(52) U.S. Cl. CPC A61B 5/1106; A61B 5/7455; A61B 5/0205; A61B 5/0402; A61B 5/6823; A61B 5/6824; A61B 5/7282; A61B 5/7405; A61B 5/01; A61B 5/742; A61B 5/02416; A61B 5/0245; A61B 5/021; A61B 5/0816; A61B 5/14542; A61B 5/4821; A61B 2562/0219

(57) ABSTRACT

An arrangement and device for determining a state of consciousness of a patient includes an accelerometer or other feedback detection device for detecting movements or other responses of the patient. In addition the arrangement and device includes visual, auditory and physical stimulus generators, and are configured to emit of a visual, auditory and/or physical stimulus for a given time period. In addition a response signal is provided of the patient to the emitted stimulus, where said response signal is recorded using said accelerometer or said feedback detection device. In addition the response of the patient is determined and the patient is labelled as alert (A), responsive to verbal or auditory stimulus (V), responsive to pain or physical stimulus (P) or unconscious patient (U) based on the response.

### TECHNICAL FIELD OF THE INVENTION

The invention relates to a device and a method for continuously and intermittently monitoring the response of a human being to a given stimuli in order to determine the state of consciousness, especially decreased consciousness due to a medical condition and depth of sleep.

### BACKGROUND OF THE INVENTION

Decreased consciousness together with altered respiratory rate are two of the most important factors predicting the development of serious adverse events in hospitalized patients. The causes of decreased consciousness are diverse including infections (especially meningitis/encephalitis), intoxication, metabolic causes (especially diabetes, respiratory causes), anoxia (especially stroke, intracranial expansions, altered cerebrovascular circulation due to intra- or extracranial causes) and intracranial bleeding (epidural, subdural, subarachnoid and intracerebral bleeding).

State of consciousness and respiratory function are closely associated since decreased respiration results in impaired gas exchange, i.e. decreased removal of carbon dioxide from the blood by lungs. The resulting accumulation of carbon dioxide in blood results in respiratory acidosis which is initially compensated by increased activity of the vasomotor center located in the brainstem resulting in hyperventilation and increased heart rate. In case these compensatory mechanisms do not succeed, the result is respiratory arrest and ultimately death. Response of the human nervous system to an external stimulus depends on the state of consciousness, for example, normally sleeping patients demonstrate spontaneous movements during sleep but they also respond to an auditory or physical stimulus by increased movement, heart rate, blood pressure and respiratory rate, while those with heavily altered consciousness do not show these responses at all. Normal sleep, especially non-rapid eye movement sleep (NREM) is characterized by a reduction in overall physiological activity, i.e. respiratory rate, heart rate, blood pressure and body temperature, all decrease during NREM sleep. Rapid eye movement-sleep (REM) is different phase characterized by more rapid, irregular and shallow breathing and limb movements, in addition to increased heart rate and blood pressure. During normal sleep, NREM and REM phases alternate every 90-110 minutes in 4-6 cycles per night.

Currently, consciousness can be evaluated by human raters using different assessment strategies such as Glasgow Coma Scale (GCS), Simplified Motor Scale (SMS) which is a simplified portion of GCS motor evaluation, FOUR Score, ACDU Score and AVPU assessment. In the AVPU assessment there are four outcomes which are rated by the level of response to a certain type of stimulus. The patient is classified as “Alert”when the patient is fully awake and responds spontaneously by opening eyes, responding to voice and has normal motor function. The classification as “Voice” includes patients that are not fully awake but respond to an auditory stimulus. “Pain” denotes patients who do not respond to auditory stimuli but respond to a painful, physical stimulus. “Unconscious” patients do not respond to any of the previous stimuli.

The problem with these assessments is that they require human resources and therefore consume limited healthcare resources and are prone to human assessment bias. Prior art has been published utilizing conventional methodology which is based on electroencephalography (EEG), electromyography (EMG), and electro-oculography (EOG). These techniques necessitate the use of multiple electrodes, significant discomfort for the patient and complex signal analysis algorithms. In addition, these methodologies cannot be used for ambulatory monitoring. A wristband based ambulatory device has previously been developed but it is based on EMG, thermistor for skin temperature monitoring and vibro-tactile stimulator. In another solution, multiple signals from pulse oximeter, forehead EEG, and temperature sensor are utilized to determine the state of consciousness. Other previous solutions are based on EEG, and EEG responsiveness. A computerized, automated acquisition of the GCS has been published but it is still unsuitable for ambulatory use since it utilizes invasive detection methods. A previous solutions based on a wristband and related accessories provides automated evaluation of consciousness and relies on motion sensors (1-D, 2-D, or 3-D accelerometers), pressure sensors in patient's bed and a video camera monitoring eye movements, microphones detecting acoustic signals or other devices.

There are however also some additional shortcomings and disadvantages relating to the known prior art. Firstly, the prior art solutions are typically focused to measure only a certain signal, whereupon the results may be unreliable. Secondly, the prior art is based on instrumentation requiring heavy signal processing, multiple sensors located on different parts of the body and therefore may either limit the mobility of the patients, cause discomfort, or interfere or tangle with the wirings or sensors of other monitoring equipment. For practical purposes, there is a great demand for a robust method capable of classifying patients to those with normal and altered state of consciousness (A vs. V, P or U; alert (A), verbal or auditory stimulus (V), pain or physical stimulus (P) and unconscious patient (U)) but no such a method currently exists. Current consumer devices monitoring sleep rely solely on passive monitoring of limb or torso movements.

### SUMMARY OF THE INVENTION

An object of the invention is to alleviate and eliminate the problems relating to the known prior art. Especially the object of the invention is to provide a simplified method and device for monitoring of state of consciousness continuously, intermittently and non-invasively.

The object of the invention can be achieved by the features of independent claims.

The invention relates to an arrangement for determining a state of consciousness of a patient according to claim 1. In addition the invention relates to a device for determining a state of consciousness of a patient according to claim 10, as well as to a method according to claim 11. Additionally the invention relates also to a computer program according to claim 12 and related feedback loops and auxiliary data inputs.

According to an embodiment of the invention the device comprises accelerometers, as well as visual, auditory and physical stimulus generators. In addition it may comprise also other sensors, such as temperature sensors. The accelerometers may be used to provide data on both movements and but also on accidental incidents such as falls. In addition, according to an example the device comprises also a data transfer interface (such as wireless interface) for transferring measurement data to a backend, wherein the more detailed determination of the state of consciousness of the patient can be executed by the backend data processing devices.

The data processing backend may comprise e.g. cloud server, any computer or mobile phone application and according to an example the backend may provide and/or send the calculated results about the state of consciousness, or an alert or the like further to an auxiliary device. The data processing backend may also provide said processed data e.g. for displaying purpose back to the wristband device or other data displaying device, such as a computer or the like in data communication network or to a smartphone of the user or any other third party device. The data may comprises e.g. information about the determined state of consciousness of the patient, such as alert (A), or response activity in response to a verbal or auditory stimulus (V), pain or physical stimulus (P) and indication of an unconscious patient (U).

The response of the patient to the stimuli may be defined for example by the following determined parameters: 1. Movement (e.g. by the accelerometer), 2. Heart rate change (pulse detector), 3. Blood pressure change (e.g. by pulse time transit measurement and suitable algorithms), 4. Temperature change (e.g. by a thermocouple), 5. SpO₂ change (e.g. by an infrared detector), and 6. Respiratory rate change. The criteria for response may be adjusted, as described elsewhere in this document. In addition it is to be noted that according to an example all of the above mentioned sensors or detectors can be incorporated into a same device, advantageously to a wristband or chest worn device.

In addition the data processing backend may also send controlling information to the device for performing measurements, such as adjusting a time interval of monitoring based e.g. on the currently determined state of consciousness. The controlling information may also comprise controlling data to provide emission of a visual, auditory and/or physical stimulus by the visual, auditory and/or physical stimulus generators of the device.

It is to be noted that the arrangement may also gather and notice other information, such as take into account patient's other biosignals, most importantly the vital signs, i.e. heart rate, blood pressure, respiratory rate, arterial blood oxygen saturation, blood oxygen saturation or SpO₂. By taking into account also these other biosignals, the medical conditions can be identified in more accurate and reliable way.

These biosignals are responsible for example for potential change in the status of consciousness (infections, intoxication, metabolic causes, respiratory causes, anoxia and bleeding). Ultimately, several clinically validated early warning scores related to these biosignals can be introduced which convert these inputs into composite risk scores which are capable of predicting serious adverse events. For example for composite early warning scores, a deep level of unconsciousness can impact the heart rate variability and respiratory rate. It is to be noted that according to an embodiment the arrangement and also the device of the invention may comprise suitable detectors and sensors as well as logics to measure, determine and manage also these other biosignals and determined said early warning scores.

In addition it is to be noted that according to an embodiment the data processing can also be implemented (at least partially) by the device. The device is advantageously implemented by a device to be worn by the patient, such as advantageously a wristband or chest worn device. The sensors are advantageously arranged so that they located at the wrist or chest or other parts of the human body. According to an embodiment, the invention relates to continuous and both passive and active (i.e. stimulus-response) monitoring when determining the state of consciousness based on information about movements, skin temperature, heart rate, respiratory rate, ECG and blood pressure.

According to an embodiment of the invention the state of consciousness is determined both passively using data on spontaneous physical activity of the patient (accelerometer) and the said biosignals from external sensors during a pre-set and adjustable time period for the monitoring, and also on physical response of a patient to a pre-set and adjustable visual, auditory and a physical stimulus (i.e. movement response, altered heart rate, blood pressure, arterial blood oxygen saturation and respiratory rate).

In addition, the object of the invention is to make it possible to gather very reliable signal for every measuring cycle taking any surrounding and environmental effect into account. The system not only modifies its function by the information acquired by the sensors and current state but also modifies the frequency of data acquisition from the sensors according to the state of consciousness. The data is received as time series waveforms from 1-D, 2-D or 3-D accelerometer sensors (with or without gyros or magnetometers), an external or integrated heart rate, blood pressure, respiratory rate, skin temperature and ECG sensors located in a device set on patient's wrist, chest or other body parts. Advantageously the measurement is done continuously and non-invasively.

According to an embodiment the present invention relates to a device and a method for assessing the level of consciousness of a person. The device assesses the state of consciousness both passively and actively. Passive assessment means that the state of consciousness is estimated by using only the monitored movement and physiological signals. Active assessment is performed by emitting stimuli of various modalities and strength levels, and at the same time monitoring the physiological vital parameters of the person and evaluating physiological responses to the provided stimuli. In addition to the automatic physiological responses, the user is able to provide a predetermined voluntary response to an observed stimulus i.e. by performing certain gesture that is recognized by the movement sensors or pressing a button. The modalities of stimuli may include but are not limited to: visual, auditory, and tactile (physical) stimulus and the monitored physiological parameters may include but are not limited to: electrocardiogram, pulse plethysmography, impedance plethysmography, and movement (accelerometers). The interval of performing the active assessment and the type and the strength of the used stimuli can be controlled by the consciousness estimate obtained with passive assessment and previous results of active assessment.

The present invention offers advantages over the known prior art, such as the possibility to determine the state of the consciousness of the patient in a reliable way and without any significant discomfort for the patient. In addition the invention makes the determination possible without requiring any human resources, whereupon limited healthcare resources can be saved and without any human assessment bias or errors. Especially the determination is possible without enormous quantity of multiple electrodes and wirings, as well as without any complex signal analysis algorithms or other monitoring equipment. Moreover the device of the invention can be used ambulatory and non-invasively.

### BRIEF DESCRIPTION OF THE DRAWINGS

Next the invention will be described in greater detail with reference to exemplary embodiments in accordance with the accompanying drawings, in which:

FIG. 1 illustrates a principle of an exemplary sequence of consciousness monitoring according to an advantageous embodiment of the invention, and

FIG. 2 illustrates an exemplary device and arrangement for determining the state of a human being according to an advantageous embodiment of the invention.

### DETAILED DESCRIPTION

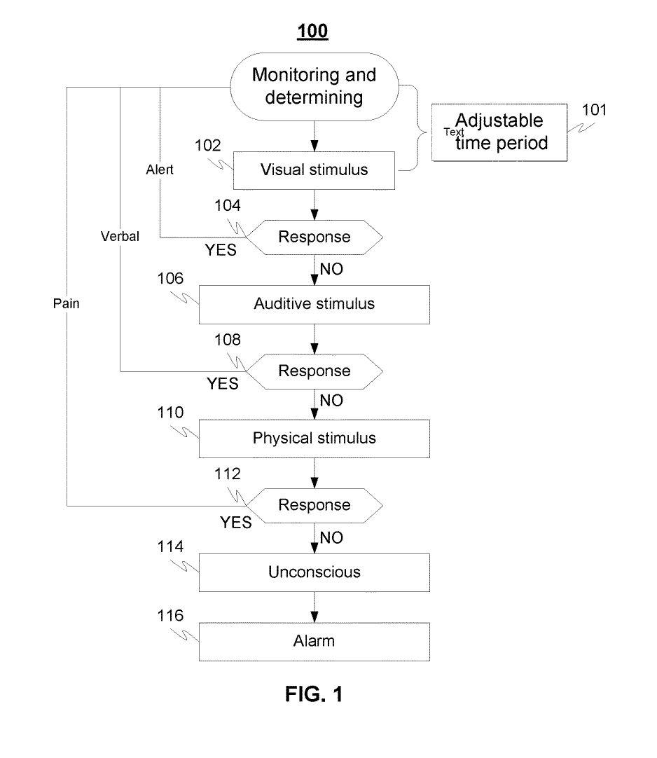

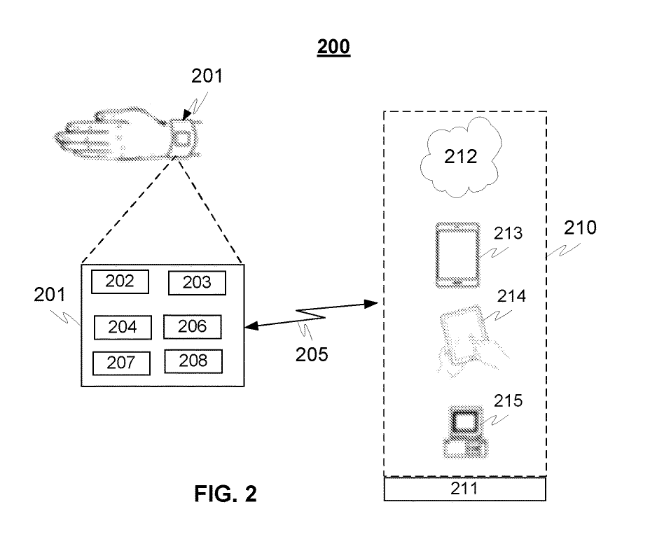

FIG. 1 illustrates a principle of an exemplary sequence 100 of consciousness monitoring according to an advantageous embodiment of the invention, and FIG. 2 illustrates an exemplary arrangement 200 and device 201 for determining the state of a human being according to an advantageous embodiment of the invention. The arrangement 200 and/or the device 201 are advantageously configured to implement the steps method 100.

The device 201 may be located at the chest, wrist or other parts of the human body. Advantageously it is a wristband device. The device 201 contains a light source 202, a sound generator 203 and a mechanical vibrator 204, which are advantageously digitally adjustable, based for example a controlling commands received from the data processing device 211 of the backend 210. In addition the device comprises a feedback or acknowledge detection device 207, such as an accelerometer or a button to be pressed as a response to the stimuli or the like. The accelerometer is configured to determine different kinds of motions, such as typical motion response to light, sound or vibrational stimulus.

The device 201 advantageously communicates wirelessly 205 and comprises thus suitable wireless, advantageously bi-directional communication devices 206. The device is capable of monitoring the physical activity, and is possibly able to detect fall or convulsions, for example based on the accelerator sensors. The device is also able to monitor by the sensors and detection devices the response of the patient and of generating a visual stimulus 102, an auditory stimulus 106 and physical stimuli (vibration, pain) 110 by the stimulus devices 202-204. The method steps are performed correspondingly.

The device or arrangement is capable of adjusting 101 the time interval of monitoring based most importantly on the currently determined state of consciousness, i.e. the time interval is decreased when the state is other than A and preferably increased if the state is A, on information from the following inputs: the motion sensors, in the device itself and also from external sensors (not shown, may also be included into the device 201 or at least they may be in data communication with the device 201) providing data on heart rate, blood pressure, arterial blood oxygen saturation and respiratory rate. According to an embodiment the device 201 may also comprise suitable sensors 208 for at least gathering necessary measuring information for determination said additional information, such as heart rate, blood pressure, arterial blood oxygen saturation and respiratory rate, for example.

Advantageously the communication between the device 201 and the backend 210 is two-way so that the arrangement is also capable of adjusting the time interval for the determination of the input signals. This way, a preset deviation of for example but not limited to heart rate, respiratory rate, blood pressure, ECG alterations, body temperature, arterial blood oxygen saturation results in altered duration of the monitoring intervals and these alterations also modify the frequency for the determination of heart rate, respiratory rate, blood pressure, ECG and SpO2. Similarly, if the accelerometers do not detect any movement for a pre-set monitoring period, the emission of the stimulus is executed. A rapid increase in acceleration may also indicate a fall or convulsions and executes an immediate sequence resulting in determination of the state of consciousness.

The data processing backend may be implemented e.g. cloud server 212, any computer or mobile phone application 213-215.

Advantageously a light stimulus emitted in step 102 is such that it exceeds the threshold to be seen by an awake patient despite having eyes closed. The appearance of all the stimuli (visual, auditory, physical) may be adjusted, i.e. they may be continuous, intermittent at a desired rate or produce sequences in a pre-determined manner, e.g. increase in strength, or with a certain waveform. The waveform and sequence of the stimulus may be adjusted e.g. according to current state and response. The patient must respond (104, 108, 112) to a stimulus in a pre-determined way which can also be adjusted and defined. The appropriate response of the patient may be increased heart rate, respiratory rate, blood pressure or increased motion detected by the accelerometers 207 or pre-instructed voluntary action of a patient to tap the device (for example twice but not necessarily limited to that action) to confirm the state.

The movement or voluntary action is detected by the accelerometers as peaks in the continuous waveform. For example, in response to a blinking light 102, the patient must tap the device or move in a pre-determined way to confirm the state A within a pre-determined time. If there is no response 104, the stimulus may be strengthened or repeated. If there is no response 104, an auditory stimulus 106 is given and the patient must respond 108 as earlier. If the patient responds 108, then the patient is classified as state V. If there is no response 108, a physical stimulus 110 is induced. If there is appropriate response 112, then the state is classified as P. If no response 112 is received, then the patient is classified as unconscious (U) 114 and an alarm sequence is executed 116. Sequentially strengthening of the stimulus enables determination of the state of consciousness and classification of the patient to a certain consciousness level, namely A-V-P-U.

The invention has been explained above with reference to the aforementioned embodiments, and several advantages of the invention have been demonstrated. It is clear that the invention is not only restricted to these embodiments, but comprises all possible embodiments within the spirit and scope of the inventive thought and the following patent claims. The features recited in dependent claims are mutually freely combinable unless otherwise explicitly stated.

It is to be noted that according to an embodiment two kinds of feedbacks can be determined, namely the state of consciousness can estimated passively by using the monitored movement and physiological signals, but also actively by emitting stimuli of various modalities and strength levels, and at the same time monitoring the physiological vital parameters of the person and evaluating physiological responses to the provided stimuli. The active feedback can be understood as an “acknowledge”, which is very convenient to determine by the accelerometer, as is described in this document in many embodiments, but can also be determined by a button to be pressed, for example. The passive feedback can be understood as a “reaction”, which is also very convenient to determine by the accelerometer, because there is no need for any action by the patient. The passive reaction may be e.g. an awaking reaction for stimulus, turning on a sleep. Alternatively or in addition to also heart rate can be determined and noticed changes in pulse as a reaction for stimulus, for example by using EKG or EEC sensors or the like.

1. An arrangement for determining a state of consciousness of a patient, wherein the arrangement comprises:
an accelerometer or other feedback detection device
visual, auditory and/or physical stimulus generators, whereupon the arrangement is configured to
emitting of a visual, auditory and/or physical stimulus by the visual, auditory and/or physical stimulus generators for a given time period,
providing a response signal of the patient to the emitted stimulus, said response signal configured to be recorded using said accelerometer or said feedback detection device,
determining the response of the patient and labelling the patient as alert (A), responsive to verbal or auditory stimulus (V), responsive to pain or physical stimulus (P) or unconscious patient (U) based on the response.

2. An arrangement of claim 1, wherein the arrangement is further configured for monitoring the patient for a pre-determined time interval, wherein the time interval is adjusted according to a current state of consciousness determined.

3. An arrangement of claim 1, wherein the arrangement is configured to provide motion data of the patient based on the accelerometer or feedback detection device measurement data, and if there is no movement for a given pre-determined duration of time, the arrangement is configured to provide said emitting of a visual, auditory and/or physical stimulus.

4. An arrangement of claim 1, wherein the accelerometer, feedback detection device and other sensors, as well as said visual, auditory and/or physical stimulus generators are to be located at the wrist or chest area of the patient.

5. An arrangement of claim 1, wherein the accelerometer data is used to provide data on both movements and but also on accidental incidents such as falls.

6. An arrangement of claim 1, wherein the arrangement is configured to emit said visual, auditory and/or physical stimulus of various modalities and strength levels, and wherein said visual, auditory and physical stimulus generators are used sequentially so that first is emitted visual stimulus, secondly auditory stimulus and third physical stimulus if there is no response for previous stimulus.

7. An arrangement of claim 1, wherein state of consciousness is estimated passively by using the monitored movement and physiological signals, and actively by emitting stimuli of various modalities and strength levels, and at the same time monitoring the physiological vital parameters of the person and evaluating physiological responses to the provided stimuli.

8. An arrangement of claim 1, wherein the arrangement is configure to gather and take into account additional information, such as patient's other biosignals, comprising information of heart rate, blood pressure, respiratory rate, arterial blood oxygen saturation, blood oxygen saturation or SpO₂, temperature, ECG, pulse plethysmography, impedance plethysmography or motion sensors comprising 1-D, 2-D or 3-D accelerometers and composite early warning scores.

9. An arrangement of claim 1, wherein the arrangement comprises a device, such as wristband or chest worn device, and backend data processing unit, whereupon the device comprised said an accelerometer, visual, auditory and/or physical stimulus generators, and data communication interface to data transmission between said device and the backend data processing unit, and wherein said device is configured to transfer said measured response signals to said backend data processing unit, and wherein said backend data processing unit is configured to label the patient as alert (A), responsive to verbal or auditory stimulus (V), responsive to pain or physical stimulus (P) or unconscious patient (U) based on the response.

10. A device for determining a state of consciousness of a patient, wherein the device comprises:
an accelerometer or other feedback detection device,
visual, auditory and/or physical stimulus generators, whereupon the device is configured to
emit of a visual, auditory and/or physical stimulus by the visual, auditory and/or physical stimulus generators for a given time period,
provide a response signal of the patient to the emitted stimulus, said response signal configured to be recorded using said accelerometer or other feedback detection device, and either
determine the response of the patient and labelling the patient as alert (A), responsive to verbal or auditory stimulus (V), responsive to pain or physical stimulus (P) or unconscious patient (U) based on the response, or
send said response signal to a backend data processing unit for said determination.

11. A method for determining a state of consciousness of a patient, wherein the method comprises:
a) emitting of a visual, auditory and/or physical stimulus by the visual, auditory and/or physical stimulus generators for a given time period,
b) providing a response signal of the patient to the emitted stimulus, said response signal configured to be recorded using said accelerometer or other feedback detection device,
c) determining the response of the patient and labelling the patient as alert (A), responsive to verbal or auditory stimulus (V), responsive to pain or physical stimulus (P) or unconscious patient (U) based on the response.

12. (canceled)

13. A non-transitory program storage device readable by a machine, tangibly embodying a program of instructions executable by the machine for performing operations, the operations comprising the method as claimed in claim 11.
